(19) United States
(12) Patent Application Publication
(10) Pub. No.: US 2014/0235911 A1
(43) Pub. Date: Aug. 21, 2014

(54) CATALYST FOR THE PREPARATION OF AROMATIC HYDROCARBONS AND USE THEREOF

(71) Applicant: Subhash Chandra Laha, Bangalore (IN)

(72) Inventor: Subhash Chandra Laha, Bangalore (IN)

(73) Assignee: SAUDI BASIC INDUSTRIES CORPORATION, Riyadh (SA)

(21) Appl. No.: 14/234,050

(22) PCT Filed: Jul. 17, 2012
(86) PCT No.: PCT/EP2012/003004

§ 371 Date: Apr. 15, 2014

(30) Foreign Application Priority Data
Jul. 21, 2011 (EP) 11005973.0

(51) Int. Cl. B01J 29/87; C07C 2/76

(52) U.S. Cl. CPC B01J 29/87; C07C 2/76; C07C 2529/87
USPC 585/417; 502/61

(57) ABSTRACT

The present invention relates to catalyst composition comprising lanthanum and gallium containing zeolite and lanthanum modified binder, wherein said lanthanum and gallium containing zeolite comprises about 0.01-0.1 wt-% lanthanum and wherein said lanthanum modified binder comprises about 0.5-2 wt-% lanthanum. Furthermore, the present invention relates to a method for preparing the catalyst composition of the present invention and a process for producing aromatic hydrocarbons comprising contacting a feedstream comprising lower alkanes with the catalyst composition of the present invention under conditions suitable for alkane aromatization.

### CROSS-REFERENCE TO RELATED APPLICATIONS

The present application is a 371 of International Application No. PCT/EP2012/003004, filed Jul. 17, 2012, which claims priority to European Application No. 11005973.0, filed Jul. 21, 2011, the contents of which are incorporated herein by reference.

The present invention relates to catalyst composition comprising lanthanum and gallium containing zeolite and lanthanum modified binder, wherein said lanthanum and gallium containing zeolite comprises about 0.01-0.1 wt-% lanthanum and wherein said lanthanum modified binder comprises about 0.5-2 wt-% lanthanum. Furthermore, the present invention relates to a method for preparing the catalyst composition of the present invention and a process for producing aromatic hydrocarbons comprising contacting a feedstream comprising lower alkanes, preferably C₂-C₆ alkanes, with the catalyst composition of the present invention under conditions suitable for alkane aromatization.

It has been previously described that that lower alkanes can be directly converted into a product stream comprising aromatic hydrocarbons using zeolite-based catalyst.

WO 2008/080517 describes a process wherein aromatic hydrocarbons are produced by contacting lower alkanes with a catalyst composition comprising a gallium containing zeolite and lanthanum modified kaolin as a binder. The nominal lanthanum load of the lanthanum modified binders of WO 2008/080517 is described to be 1 wt-%.

CN1296861 discloses a catalyst useful for hydrocarbon aromatization composed of ZSM-5 with a Si/Al mol ratio of 20-70, further comprising Ga and one metal selected from the group consisting of La, Ag, Pd, Zn and Re. The composition comprises 46-99.4 wt-% ZSM-5; 0.5-2 wt-% Ga; 0.01-2 wt-% of the metal selected from the group consisting of La, Ag, Pd, Zn and Re; and optionally up to 50 wt-% alumina. In a preferred embodiment, the composition comprises 63-99 wt-% ZSM-5; 0.8-1.6 wt-% Ga; 0.1-1 wt-% of the metal selected from the group consisting of La, Ag, Pd, Zn and Re; and optionally up to 35 wt-% alumina.

EP 0 283 212 A1 and U.S. Pat. No. 7,164,052 disclose a process for producing aromatic hydrocarbon compounds comprising contacting C2-C6 hydrocarbons with a catalyst composition comprising gallium and at least one lanthanide element, preferably lanthanum, and a zeolite, preferably MFI/ZSM-5. The zeolite catalyst of EP 0 283 212 A1 may contain from 0.2 to 1 wt-% of gallium and from 0.1 to 2, preferably 0.1 to 0.8 wt-% of rare earth, preferably lanthanum. The zeolite catalyst of U.S. Pat. No. 7,164,052 may contain 0.05 to 10 wt-% gallium and 0.01 to 10 wt-% lanthanide element and based on the total weight of the catalyst composition.

A drawback of conventional zeolite-based catalyst useful in the aromatization of lower alkanes is that the selectivity for aromatics is relatively low. Furthermore, it was found that catalyst activity of conventional zeolite-based catalyst in alkane aromatization process is reduced over time.

It was an object of the present invention to provide a catalyst useful for the aromatization of lower alkanes, having an improved selectivity for useful aromatic hydrocarbons, such as BTX, and which has a more stable catalyst activity.

The solution to the above problem is achieved by providing the embodiments as described herein below and as characterized in the claims. Accordingly, the present invention provides a catalyst composition comprising: lanthanum (La) and gallium (Ga) containing zeolite (La/Ga/zeolite); and lanthanum (La) modified binder (La/binder), wherein said La/Ga/zeolite comprises about 0.01-0.1 wt-% La with respect to the total La/Ga/zeolite and wherein said La/binder comprises about 0.5-2 wt-% La with respect to the total La/binder.

In the context of the present invention, it was found that in an alkane aromatization process, a high alkane conversion rate of 50-70 mol-% and high selectivity for BTX of 50-65 mol-% can be achieved when using the bound La/Ga/zeolite catalyst of the present invention, wherein the La/Ga/zeolite catalyst component comprises about 0.01-0.1 wt-% La and wherein the La/binder comprises about 0.5-2 wt-% La. Moreover, it was found that the stability of the catalyst against deactivation is remarkably improved to continuous runs of up to 100-150 hours when compared to conventional bound La-comprising zeolite catalysts.

Preferably, the catalyst composition comprises La/Ga/zeolite comprising about 0.02-0.09 wt-% La with respect to the total La/Ga/zeolite. More preferably, the catalyst composition comprises La/Ga/zeolite comprising about 0.03-0.08 wt-% La with respect to the total La/Ga/zeolite. Most preferably, the catalyst composition comprises La/Ga/zeolite comprising about 0.04-0.07 wt-% La with respect to the total La/Ga/zeolite.

Preferably, the catalyst composition comprises La/Ga/zeolite comprising about 0.2-2 wt-% Ga with respect to the total La/Ga/zeolite. Most preferably, the catalyst composition comprises La/Ga/zeolite comprising about 0.5-1.5 wt-% Ga with respect to the total La/Ga/zeolite. Selecting the preferred Ga content further improves conversion and BTX selectivity.

The catalyst composition of the present invention comprises a binder that is modified with La (La/binder). Any conventional catalyst binder that can be modified with La may be used. It is well within the scope of the skilled person to select a suitable binder; see Otterstedt et al (1998). Preferably, the binder is selected from the group consisting of alumina, silica, kaolin, bohemite and bentonite. More preferably, the binder is kaolin. The catalyst composition of the present invention preferably comprises about 5-50 wt-% La/binder with respect to the total catalyst composition.

The catalyst composition comprises zeolite. As used herein, the term “zeolite” or “aluminosilicate zeolite” relates to an aluminosilicate molecular sieve. These inorganic porous materials are well known to the skilled person. An overview of their characteristics is for example provided by the chapter on Molecular Sieves in Kirk-Othmer Encyclopedia of Chemical Technology, Volume 16, p 811-853; in Atlas of Zeolite Framework Types, 5^{th} edition, (Elsevier, 2001). Preferably, the zeolite is a medium pore size aluminosilicate zeolite. Most preferably the zeolite is ZSM-5 zeolite, which is a well-known zeolite having MFI structure. Other suitable zeolites include, but are not limited to, MCM-22 and ZSM-11. The term “medium pore zeolite” is commonly used in the field of zeolite catalysts. Accordingly, a medium pore size zeolite is a zeolite having a pore size of about 5-6 Å. Suitable medium pore size zeolites are 10-ring zeolites. i.e. the pore is formed by a ring consisting of 10 SiO₄ tetrahedra. Zeolites of the 8-ring structure type are called small pore size zeolites; and those of the 12-ring structure type, like for example beta zeolite, are also referred to as large pore sized. In the above cited Altlas of Zeolite Framework Types various zeolites are listed based on ring structure.

The zeolite of the present invention may be dealuminated. Preferably, the silica (SiO₂) to alumina (Al₂O₃) molar ratio of the ZSM-5 zeolite is in the range of about 10-200. Means and methods to obtain dealuminated zeolite are well known in the art and include, but are not limited to the acid leaching technique; see e.g. Post-synthesis Modification I; Molecular Sieves, Volume 3; Eds. H. G. Karge, J. Weitkamp; Year (2002); Pages 204-255. In the context of the present invention it was found that using a dealuminated zeolite having a SiO₂ to Al₂O₃ molar ratio of 10-200 improves the performance/stability of the catalyst. Means and methods for quantifying the SiO₂ to Al₂O₃ molar ratio of a dealuminated zeolite are well known in the art and include, but are not limited to AAS (Atomic Absorption Spectrometer) or ICP (Inductively Coupled Plasma Spectrometry) analysis.

It is preferred that the zeolite is in the hydrogen form: i.e. having at least a portion of the original cations associated therewith replaced by hydrogen. Methods to convert an aluminosilicate zeolite to the hydrogen form are well known in the art. A first method involves direct ion exchange employing an acid. A second method involves base-exchange using ammonium salts followed by calcination.

In a further aspect of the present invention a method for preparing a catalyst composition is provided. Accordingly, the present invention provides a method for preparing the catalyst composition as described herein comprising the steps of:
(i) preparing gallium and lanthanum containing zeolite (La/Ga/zeolite) comprising about 0.01-0.1 wt-% La with respect to the total La/Ga/zeolite;
(ii) preparing lanthanum modified binder (La/binder) comprising about 0.5-2 wt-% La with respect to the total La/binder; and
(iii) combining said La/Ga/zeolite and said La/binder.

Preferably, La/Ga/zeolite is prepared in the above defined La/Ga/zeolite preparation step (i) by ion-exchange and/or impregnation with a solution comprising gallium (Ga) salt and a solution comprising lanthanum (La) salt. Preferably, the Ga-salt solution and the La-salt solution are aqueous solutions. The La/binder is preferably prepared in the above defined La/binder preparation step (ii) by impregnation of the binder with a solution comprising lanthanum (La) salt. A preferred Ga salt used to prepare the solution is gallium(III) nitrate. A preferred La salt used to prepare the solution is lanthanum(III) nitrate. Preferably, the solution comprising lanthanum (La) salt used in step (i) comprises about 0.001-0.01 M La, more preferably 0.002-0.006 M La and wherein the solution comprising lanthanum (La) salt used in step (ii) comprises about 0.01-0.1 M La, more preferably 0.02-0.06 M La. The solution comprising lanthanum (La) salt used in step (i) comprises a higher concentration of La than the solution comprising lanthanum (La) salt used in step (ii).

For incipient wetness or wetness impregnation, minimum amount of solvent, preferably water, is used to dissolve the metal salt which as aqueous solution of the salt is just sufficient to soak the catalyst or the binder and prepare a dry thick paste. Since the lanthanum loading on the binder is 10-20 times to that of the concentration on the catalyst, different concentrations of the La-solution are required for effective impregnation of lanthanum in the catalyst as well as in the binder.

The final catalyst composition may be prepared by mixing the La/Ga/zeolite component and the La/binder component at a specific weight ratio, followed by pelletizing the mixture. Alternatively, the final catalyst composition may be prepared by separately preparing pelletized particles of the La/Ga/zeolite component and pelletized particles of the La/binder and mixing the two components (in particle forms) at a specific weight ratio.

In a further aspect of the present invention the catalyst composition of the present invention as obtainable by the method for preparing a catalyst composition as described herein is provided. Accordingly, the present invention provides a catalyst composition obtainable by the method comprising the steps of:
(i) preparing gallium and lanthanum containing zeolite (La/Ga/zeolite) comprising about 0.01-0.1 wt-% La with respect to the total La/Ga/zeolite;
(ii) preparing lanthanum modified binder (La/binder) comprising about 0.5-2 wt-% La with respect to the total La/binder; and
(iii) combining said La/Ga/zeolite and said La/binder.

In a further embodiment of the present invention, a process for producing a product stream comprising aromatic hydrocarbons wherein the catalyst composition as described herein is contacted with a feedstream comprising lower alkanes at conditions suitable for alkane aromatization. The process of the present invention is inter alia characterized in that the product stream produced by the herein described process after 24 hours on stream comprises at least 55 wt-% of benzene, toluene and xylene (BTX), more preferably at least 58 wt-% BTX and most preferably at least 60 wt-% BTX.

The lower alkanes that are preferably comprised in the feedstream are C₂-C₆ alkanes (i.e. alkanes having 2-6 carbon atoms), more preferably C₃-C₄ alkanes.

The terms “aromatic hydrocarbon” is very well known in the art. Accordingly, the term “aromatic hydrocarbon” relates to cyclically conjugated hydrocarbon with a stability (due to delocalization) that is significantly greater than that of a hypothetical localized structure (e.g. Kekulé structure). The most common method for determining aromaticity of a given hydrocarbon is the observation of diatropicity in the ¹H NMR spectrum. Preferably, the aromatic hydrocarbons produced in the process of the present invention are aromatic hydrocarbons having between 6 and 12 carbon atoms (C6-C12 aromatics). More preferably, the hydrocarbons produced in the process of the present invention are BTX, which is a commonly known abbreviation of a mixture of benzene, toluene and xylenes.

The process conditions useful in the process of the present invention, also described herein as “alkane aromatization conditions”, can be easily determined by the person skilled in the art; see O'Connor, Aromatization of Light Alkanes. Handbook of Heterogeneous Catalysis Wiley-VCH 2008, pp 3123-3133. Accordingly, the process of the present invention is preferably performed at a temperature of about 450-600° C. and a weight hourly space velocity (WHSV) of about 0.5-5.0.

Furthermore, a process for producing a product stream comprising aromatic hydrocarbons is provided wherein the catalyst composition of the present invention is contacted with a feedstream comprising lower alkanes, preferably C₂-C₆ alkanes, at conditions suitable for alkane aromatization, wherein said catalyst composition is prepared by the method as described herein comprising the steps of:
(i) preparing gallium and lanthanum containing zeolite (La/Ga/zeolite) comprising about 0.01-0.1 wt-% La with respect to the total La/Ga/zeolite;
(ii) preparing lanthanum modified binder (La/binder) comprising about 0.5-2 wt-% La with respect to the total La/binder; and
(iii) combining said La/Ga/zeolite and said La/binder.

### MODE(S) FOR CARRYING OUT THE INVENTION

The present invention will now be more fully described by the following non-limiting Examples.

### Preparation of Ga/ZSM-5 Zeolite

0.5714 g gallium nitrate was dissolved in 200 ml demineralised water in a 3-neck round bottom flask. 10 gm of dry ZSM-5 in NH₄ form, having a Si/Al molar ratio of 25 (equals silica to alumina molar ratio of 50) was added. The mixture was heated to 90-95° C. and stirred at 300 rpm for 4 hrs. The Ga-exchanged ZSM-5 was filtered, washed with 2 litres of demineralised water and dried in air oven at 120° C. for over night. The Ga content on the zeolite was determined by AAS and ICP to be around 1 wt %. This procedure can be applied to prepare Ga exchanged ZSM-5 with other Si/Al ratios.

### Preparation of La/Ga/ZSM-5 Zeolite

0.0156 g lanthanum nitrate hexahydrate was dissolved in 10 ml demineralised water. 10 gm of dry Ga/ZSM-5 was taken on a Petri dish and lanthanum nitrate solution was added dropwise to the Ga/ZSM-5 and mixed well to make a thick homogenous paste. The paste was dried in air oven at 120° C. for over night and then calcined at 550° C. in zero air with the flow of 100 ml/min for 4 hrs. The La content on the zeolite was determined by ICP to be around 0.05 wt %. This procedure can be applied to prepare La/Ga/ZSM-5 catalysts with different La-composition.

### Preparation of Support Materials (Binders)

0.3118 g lanthanum nitrate hexahydrate was dissolved in 15-20 ml demineralised water. 10 gm of kaolin was taken on a Petri dish and lanthanum nitrate solution was slowly added to kaolin to make a thick homogenous paste. The paste was dried in air oven at 120° C. for over night and then calcined at 550° C. in zero air with the flow of 100 ml/min for 4 hrs. The La content on the zeolite was determined by ICP to be around 1.0 wt %. This procedure can be applied to prepare La/kaolin binders with different La-composition. Further, this procedure can be applied to prepare different La/binders.

Preparation of La-Containing Catalyst Using Blended Ga/ZSM-5 (Zeolite) and Kaolin (Binder)

0.0156 g lanthanum nitrate hexahydrate was dissolved in 10 ml demineralised water. The mixture of 6.67 gm of dry 1.0 wt-% Ga/ZSM-5 and 3.33 g of kaolin was taken on a Petri dish and lanthanum nitrate solution was slowly added to the mixture. A thick homogenous paste was prepared. The paste was dried in air oven at 120° C. for over night and then calcined at 550° C. in zero air with the flow of 100 ml/min for 4 hrs. The La-content on the zeolite/binder composition was determined by ICP to be around 0.05 wt %. This procedure can be applied to prepare La-containing different zeolite/binder catalyst composition with different La-concentration. These catalysts are represented as X wt-% La-loaded (1 wt-% Ga/ZSM-5+kaolin).

### Preparation of Catalyst Particles

A number of catalyst compositions comprising different zeolites and binder supports were prepared in particle form by mixing thoroughly the zeolite and the binder support in 2:1 ratio. The mixture was pressed at 10 ton pressure to make pellets. The pressed catalyst compositions were crushed, sieved. The fraction containing particles from 0.25 to 0.5 mm and the fraction containing particles from 0.5 to 1.00 mm particles were selected for further use.

### Catalyst Testing

Two grams catalyst particles (particle size 0.25-0.5 mm) were loaded in a down flow fixed bed micro catalytic reactor and pre-treated in the following way:

Step 1: Exposed for 1 h to moisture-free air flow of 25 ml/min at 600° C.;

Step 2: Exposed for 1 h to 50 ml/min hydrogen flow at 525° C.

After the pre-treatment, propane was fed to the bed at a rate of 23.33 ml/min. The temperature of the catalyst bed before start of propane flow was 525° C. The Weight Hourly Space Velocity (WHSV) was 1.4 h⁻¹.

Unconverted propane and formed products were analysed by an on-line Gas Chromatograph, separation column Petrocol DH 50.2, using a Flame Ionization Detector.

The provided values have been calculated as follows:

### Conversion:

An indication of the activity of the catalyst was determined by the extent of conversion of the propane or for more active catalysts by the extent of volume reduction of the reagent gases (using nitrogen as internal standard). The basic equation used was:

Conversion %=Moles of propaneᵢₙ−moles of propaneₒᵤₜ/moles of propaneᵢₙ*100/1

### Selectivity

First of all, the varying response of the detector to each product component was converted into % v/v by, multiplying them with online calibration factors. Then these were converted into moles by taking account the flow out of internal standard, moles of feed in and time in hours. Moles of each product were converted into mole-% and selectivity-% was measured by taking carbon numbers into account.

### Yield

The yield of given process product was calculated by multiplying the conversion with the fraction of selectivity.

After the reaction the catalyst was regenerated in the following way:
Step 1: Exposed for 4 h in nitrogen gas (270 ml/min) with 2 vol. % of moisture-free air at 540° C.;
Step 2: The reactor was cooled to 150° C., start passing steam with nitrogen for 30 min (N₂ flow=50 ml/min, Water flow=0.0021 ml/min). This step is optional and was carried out once after five cycles (approx.)
Step 3: Increased the reactor temperature up to 525° C. with nitrogen gas (76 ml/min)
Step 4: Exposed for 30 min to 50 ml/min hydrogen flow at 525° C.

After the regeneration of the catalyst, propane was fed to the bed at a rate of 23.33 ml/min and propane aromatization reaction was continued.

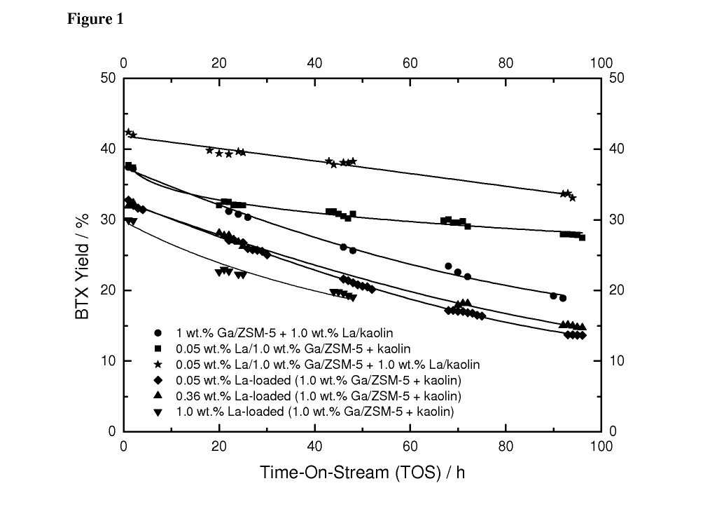

FIG. 1 is a graphical representation of Time-On-Stream (TOS) versus BTX yield for different catalysts and catalyst compositions.

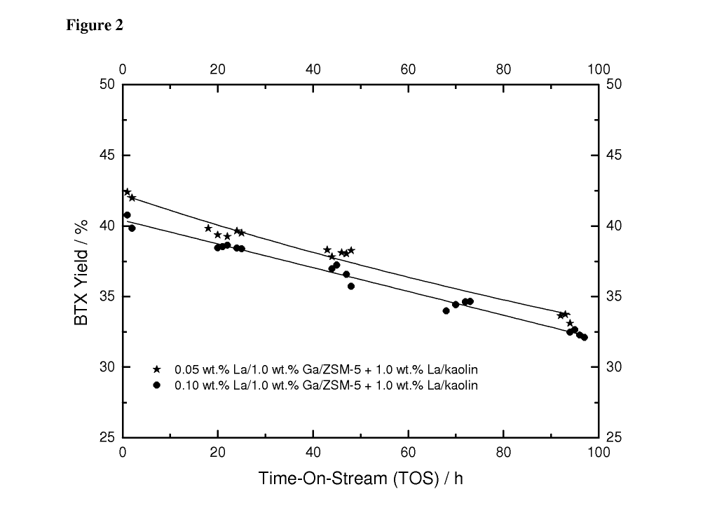

FIG. 2 is a graphical representation of Time-On-Stream (TOS) versus BTX yield for different La/Ga/ZSM-5 catalysts.

FIG. 1 provides a comparison of propane aromatization with different catalysts and catalyst compositions (Reaction temperature=525° C., Pressure=1 atmosphere, WHSV=1.4 h⁻¹). It is demonstrated that only when La is present in variable amount in the active component (Ga/ZSM-5) and the binder (kaolin), prepared separately by sequential doses and the final catalyst composition prepared by physical mixing of the two solid components, the final catalyst produced desired (higher) BTX yield and also showed high resilience against deactivation of the catalyst for a continuous reaction run of about 100 hours. Active component to binder ratio is considered as 2:1 (wt-/wt-) for the final catalysts presented below.

FIG. 2 provides a comparison of BTX yield on propane aromatization for different La/Ga/ZSM-5 catalysts (Reaction temperature=525° C., Pressure=1 atmosphere, WHSV=1.4 h⁻¹).

TABLE 1
Effect of La and its concentration on the catalytic performance for propane
aromatization reaction.

| | | | | Product distribution | | | |
|---|---|---|---|---|---|---|---|
| | | | BTX | (selectivity) (%) | | | |
| | Time | Propane | Yield | | | | C₉₊ |
| Catalysts | (h) | Convn. (% | (%) | C₁ + C₂ | C₄₊ | BTX | aromatics |
| 1% Ga/ZSM-5 + | 24 | 55.6 | 32.3 | 30.5 | 3.3 | 58.1 | 8.1 |
| 1% La/kaolin (2:1) | | | | | | | |
| (Comparative) | | | | | | | |
| 0.03% La/1% Ga/ZSM-5 + | 24 | 63.9 | 38.0 | 30.1 | 2.1 | 59.4 | 8.4 |
| 1% La/kaolin (2:1) | | | | | | | |
| 0.05% La/1% Ga/ZSM-5 + | 24 | 65.9 | 39.7 | 28.1 | 2.1 | 60.2 | 9.6 |
| 1% La/kaolin (2:1) | | | | | | | |
| 0.10% La/1% Ga/ZSM-5 + | 24 | 63.6 | 38.4 | 28.3 | 2.3 | 60.4 | 9.0 |
| 1% La/kaolin (2:1) | | | | | | | |
| 0.36% La/1% Ga/ZSM-5 + | 24 | 49.2 | 26.9 | 34.8 | 3.9 | 54.7 | 6.6 |
| 1% La/kaolin (2:1) | | | | | | | |
| (Comparative) | | | | | | | |

TABLE 2
Effect of binders on propane aromatization using catalyst composition comprising
0.05% La/1% Ga-HZSM-5 (25) + 1% La/binder (2:1) as principal components.

| | | Propane | BTX | | | | |
|---|---|---|---|---|---|---|---|
| | Time | Convn. | Yield | Product Distribution (selectivity) (%) | | | |
| Binders | (h) | (%) | (%) | C₁ + C₂ | C₄₊ | BTX | C₉₊ aromatics |
| 1% La/Kaolin | 1 | 71.9 | 42.4 | 28.3 | 1.6 | 59.0 | 11.1 |
| 1% La/Boehmite | 1 | 69.6 | 39.5 | 30.7 | 1.8 | 56.8 | 10.7 |
| 1% La/Bentonite | 1 | 50.9 | 32.2 | 33.4 | 5.1 | 45.5 | 16.0 |

TABLE 3
Effect of different feed (reactant) on the catalytic performance for propane
aromatization reaction using catalyst composition comprising 0.05% La/1% Ga-HZSM-
5 (25) + 1% La/kaolin (2:1) as principal components.

| | | Propane/ | BTX | Product distribution (selectivity) (%) | | | | |
|---|---|---|---|---|---|---|---|---|
| | Time | Butane/LPG | Yield | | | | | C₉₊ |
| Feed | (h) | Convn. (%) | (%) | C₁ + C₂ | C₃ | C₄₊ | BTX | aromatics |
| Propane | 1 | 71.9 | 42.4 | 28.3 | — | 1.6 | 59.0 | 11.1 |
| Butane | 1 | 71.5 | 41.8 | 19.4 | 19.7 | 4.5 | 49.9 | 6.5 |
| LPG (Propane: | 1 | 55.0 | 32.9 | 30.8 | — | 1.3 | 59.9 | 8.0 |
| Butane = 70:30) | | | | | | | | |

TABLE 4
Comparison of reproducibility studies of catalysts for propane aromatization
reaction with catalyst composition comprising 0.05% La/1% Ga-HZSM-5 (25) + 1%
La/kaolin (2:1) as principal components

| | | | BTX | | | | |
|---|---|---|---|---|---|---|---|
| | Time | Propane | Yield | Product distribution (selectivity) (%) | | | |
| Catalysts | (h) | Convn.(% | (%) | C₁ + C₂ | C₄₊ | BTX | C₉₊ aromatics |
| Batch 1 | 21 | 65.1 | 38.5 | 28.3 | 2.1 | 59.1 | 10.5 |
| Batch 2 | 20 | 65.4 | 38.7 | 28.2 | 2.4 | 59.2 | 10.2 |
| Batch 3 | 20 | 66.6 | 37.6 | 27.8 | 1.9 | 56.4 | 13.9 |
| Batch 4 | 20 | 66.1 | 39.3 | 29.2 | 2.0 | 59.5 | 9.3 |
| Batch 5 | 20 | 66.0 | 36.6 | 33.4 | 2.5 | 55.5 | 8.6 |

TABLE 5
Comparison of catalysts stability studies for propane aromatization
reaction using catalyst composition comprising 0.05% La/1% Ga-HZSM-5 (25) + 1%
La/kaolin (2:1) as principal components

| | | | BTX | | | | |
|---|---|---|---|---|---|---|---|
| | Time | Propane | Yield | Product distribution (selectivity) (%) | | | |
| No. of Cycle | (h) | Convn. (% | (%) | C₁ + C₂ | C₄₊ | BTX | C₉₊ aromatics |
| Cycle 1 | 48 | 63.43 | 38.3 | 28.86 | 2.35 | 60.32 | 8.48 |
| Cycle 2 | 48 | 55.76 | 33.4 | 30.76 | 2.70 | 59.90 | 6.65 |
| Cycle 3 | 48 | 59.77 | 36.1 | 27.81 | 2.56 | 60.35 | 9.28 |
| Cycle 4 | 48 | 61.24 | 36.4 | 29.25 | 2.44 | 59.36 | 8.95 |
| Cycle 5 | 48 | 59.29 | 35.9 | 27.20 | 2.77 | 60.58 | 9.46 |

1. A catalyst composition comprising: lanthanum (La) and gallium (Ga) containing zeolite (La/Ga/zeolite) and lanthanum (La) modified binder (La/binder), wherein said La/Ga/zeolite comprises about 0.01-0.1 wt-% La with respect to the total La/Ga/zeolite and wherein said La/binder comprises about 0.5-2 wt-% La with respect to the total La/binder.

2. The catalyst composition according to claim 1, wherein the La/Ga/zeolite comprises about 0.2-2 wt-% Ga with respect to the total La/Ga/zeolite.

3. The catalyst composition according to claim 1, wherein the binder is selected from the group consisting of alumina, silica, kaolin, bohemite and bentonite.

4. The catalyst composition according to claim 3, wherein the binder is kaolin which has not been acid treated.

5. The catalyst composition according to claim 1, wherein the catalyst composition comprises about 5-50 wt-% La/binder with respect to the total catalyst composition.

6. The catalyst composition according to claim 1, wherein the zeolite is ZSM-5 zeolite.

7. The catalyst composition according to claim 6, wherein the silica to alumina molar ratio of the ZSM-5 zeolite is in the range of about 10-200.

8. A method for preparing a catalyst composition, comprising:
(i) preparing gallium and lanthanum containing zeolite (La/Ga/zeolite) comprising about 0.01-0.1 wt-% La with respect to the total La/Ga/zeolite;
(ii) preparing lanthanum modified binder (La/binder) comprising about 0.5-2 wt-% La with respect to the total La/binder; and
(iii) combining said La/Ga/zeolite and said La/binder.

9. The method of preparing the catalyst composition according to claim 8, wherein in step (i) the La/Ga/zeolite is prepared by ion-exchange and/or impregnation with a solution comprising gallium (Ga) salt and a solution comprising lanthanum (La) salt.

10. The method of preparing the modified binder of the catalyst composition according to claim 8, wherein in step (ii) the La/binder is prepared by impregnation of the binder with a solution comprising lanthanum (La) salt.

11. The method according to claim 10, wherein the aqueous solution of lanthanum (La) salt used in step (i) comprises about 0.001-0.01 M, and wherein the aqueous solution of lanthanum (La) salt used in step (ii) comprises about 0.01-0.1 M La.

12. A process for producing a product stream comprising aromatic hydrocarbons wherein a catalyst composition is contacted with a feedstream comprising lower alkanes at conditions suitable for alkane aromatization;
wherein the catalyst composition comprises lanthanum (La) and gallium (Ga) containing zeolite (La/Ga/zeolite) and lanthanum (La) modified binder (La/binder), wherein said La/Ga/zeolite comprises about 0.01-0.1 wt-% La with respect to the total La/Ga/zeolite and wherein said La/binder comprises about 0.5-2 wt-% La with respect to the total La/binder.

13. The process according to claim 12, wherein the product stream comprises benzene, toluene and xylenes.

14. The process according to claim 12, wherein the lower alkanes are C₂-C₆ alkanes.

15. The process according to claim 12, wherein the process is performed at a temperature of about 450-600° C. and a weight hourly space velocity (WHSV) of about 0.5-5.0.
